(19) United States
(12) Patent Application Publication
(10) Pub. No.: US 2010/0304455 A1
(43) Pub. Date: Dec. 2, 2010

(54) ETHANOL PRODUCING PROCESS AND APPARATUS

(75) Inventors: Hiroyuki Inoue, Hiroshima (JP); Chiaki Kitao, Hiroshima (JP); Shinichi Yano, Hiroshima (JP); Shigeki Sawayama, Hiroshima (JP); Takashi Endo, Hiroshima (JP); Tetsuro Nishimoto, Hiroshima (JP); Naohiro Fujikawa, Hiroshima (JP)

Correspondence Address: MORRISON & FOERSTER LLP, 425 MARKET STREET, SAN FRANCISCO, CA, 94105-2482, US

(73) Assignees: National Institute of Advanced Industrial Science and Technology, Chiyoda-ku (JP); Juon Co., Ltd., Hiroshima-shi (JP)

(21) Appl. No.: 12/445,735

(22) PCT Filed: Oct. 11, 2007
(86) PCT No.: PCT/JP2007/069859

§ 371 Date: Apr. 15, 2009

(30) Foreign Application Priority Data
Oct. 16, 2006 (JP) 2006-281224

(51) Int. Cl. C12P 7/10; C12M 1/02

(52) U.S. Cl. 435/165; 435/303.3

(57) ABSTRACT

The present invention relates to a process for producing ethanol by carrying out the following steps: performing enzymatic saccharification of pre-treated lignocellulosic biomass in a reaction system; performing ethanol fermentation of fermentable sugars obtained from the saccharified pre-treated lignocellulosic biomass in the same reaction zone as the enzymatic saccharification; distilling ethanol directly off from a reaction treatment liquid in the reaction zone, so as to recover the ethanol. The process suitably uses an ethanol producing apparatus including: one reaction vessel having a biomass raw material inlet, a diastatic enzyme inlet, and a fermentation microorganism inlet; heating means for adjusting a temperature inside the reaction vessel; pH controlling means for controlling pH inside the reaction vessel; stirring means for stirring a liquid inside the reaction vessel; and distilling means for distilling the liquid inside the reaction vessel, which distilling means is directly connected to the reaction vessel. Hence, the present invention enables efficient production of ethanol from the lignocellulosic biomass with simple operations.

### TECHNICAL FIELD

The present invention relates to a process for efficiently producing ethanol, particularly fuel ethanol or industrial ethanol, by use of biomass (particularly lignocellulosic biomass) as raw material, and an apparatus to be used in such a process.

### BACKGROUND ART

Ethanol, which is a constituent of various alcohol drinks, has also been broadly used as industrial raw material and solvents. Furthermore, due to progression of depletion in fossil fuels such as coal, petroleum, natural gas and the like, and due to a tendency to restrict use of the fossil fuels since the fossil fuels are one generation source of CO₂ which is a cause of global warming, ethanol has been recognized as a liquid fuel which replaces the fossil fuels.

This fuel ethanol is produced mainly by fermentation, by use of, as raw material, biomass that is available as natural resources in mass amounts. This method is usually carried out via the following procedures: (i) a first step in which diastatic enzyme is added to the raw material so as to cause enzymatic saccharification, and an obtained reaction product is separated so as to obtain a saccharide-containing solution to be sent to a second step; (ii) the second step in which an ethanol-fermentable microorganism is added to the solution so as to carry out ethanol fermentation, and an obtained reaction product is separated so as to obtain an ethanol-containing aqueous solution to be sent to a distillation column; and (iii) the aqueous solution is distilled in the distillation column, and if necessary, further concentrated so as to recover concentrated ethanol (see Patent Literature 1).

FIG. 2 is an explanatory view of steps of such a conventional process for producing ethanol. As shown in FIG. 2, first, the biomass raw material is put into an enzymatic saccharification apparatus, and the diastatic enzyme is added so as to carry out the enzymatic saccharification step (in a case where the raw material is lignocellulosic biomass, the enzymatic saccharification process is carried out under a temperature condition in a range of 40° C. to 50° C., and in a case where the raw material is starch biomass, the enzymatic saccharification process is carried out under a temperature condition in a range of 80° C. to 95° C.). An enzymatically-saccharified liquid generated in this enzymatic saccharification step is subjected to a separation step, and thereafter is poured into a fermentation apparatus. The ethanol-fermentable microorganism such as yeast is added to the fermentation apparatus, so as to carry out the ethanol fermentation step at a temperature in a range of 25° C. to 35° C. Ethanol-fermented liquid that is generated in the ethanol fermentation step is subjected to a separation step, and subsequently sent to the distillation apparatus. In the distillation apparatus, the ethanol-fermented liquid thus separated is subjected to a distillation step at a temperature in a range of 90° C. to 100° C. so as to recover ethanol. This ethanol is further concentrated as desired, and is recovered as ethanol having a concentration of 15% to 95% by volume.

The conventional process carries out a plurality of procedures in separate apparatuses. Because of this, the following disadvantages occur: (1) operations become complicated, and energy is lost during the processes; (2) separation operations are required for each step, which causes loss of intermediate products each time, thereby resulting in a decrease in yield of ethanol; (3) raw material concentration in the enzymatic saccharification requires to be low so as to prevent clogging of lines that connect the apparatuses, thereby causing production efficiency to decrease; (4) since concentration of the ethanol thus recovered upon fermentation is only around 5% by volume, in order to obtain high-concentrated ethanol, great burden is given to the distillation apparatus because of the increase in liquid amount that is applied to the distillation apparatus (see Patent Literature 1), which hence require carrying out concentration by use of various separation films (see Patent Literature 2, 3, and 4); and (5) in order to provide various apparatuses, equipment is required to be in large scale.

Patent Literature 1

Japanese Patent Application Publication, Tokukaihei, No. 11-169188 A (Publication Date: Jun. 29, 1999)

Patent Literature 2

Japanese Patent Application Publication, Tokukaisho, No. 57-136905 A (Publication Date: Aug. 24, 1982)

Patent Literature 3

Japanese Patent Application Publication, Tokuhyohei, No. 2-502634 A (Publication Date: Aug. 23, 1990)

Patent Literature 4

Japanese Patent Application Publication, Tokukaihei, No. 5-245345 (Publication Date: Sep. 24, 1993)

### SUMMARY OF INVENTION

The present invention is accomplished for a purpose of providing (i) a process for producing ethanol, which overcomes the disadvantages of the conventional process, that is easily operated and can efficiently produce ethanol from biomass (particularly lignocellulosic biomass), and (ii) a tightly-assembled apparatus to be used in such a process for producing ethanol.

As a result of conducting various sorts of diligent studies of processes for producing ethanol by use of various biomass raw material, the inventors of the present invention found that by performing saccharification and ethanol fermentation of pre-treated lignocellulosic biomass in one reaction zone (in other words, “in one reaction vessel”), and directly distilling and recovering ethanol from a fermented product, it is possible to simplify operation, reduce loss in the intermediate processing steps, and produce ethanol efficiently. The present invention is accomplished based on this finding. Namely, the present invention includes inventions as follows.

An ethanol producing process according to the present invention includes the steps of: performing enzymatic saccharification of pre-treated lignocellulosic biomass in a reaction system; performing ethanol fermentation of fermentable sugars obtained from the saccharified pre-treated lignocellulosic biomass in the same reaction zone as the enzymatic saccharification; and distilling ethanol directly off from a reaction treatment liquid in the reaction zone, so as to recover the ethanol.

The ethanol producing process according to the present invention may also be a method carried out such that the enzymatic saccharification of the pre-treated lignocellulosic biomass is carried out in the presence of diastatic enzyme at a temperature in a range of 30° C. to 60° C., the ethanol fermentation of fermentable sugars obtained from the saccharified pre-treated lignocellulosic biomass is carried out in the presence of an ethanol-fermentable microorganism at a temperature in a range of 20° C. to 40° C. in the same reaction zone as the enzymatic saccharification, and the ethanol distillation is carried out under normal pressure or reduced pressure at a temperature in a range of 80° C. to 110° C.

Meanwhile, an ethanol producing apparatus according to the present invention includes: one reaction vessel having a biomass raw material inlet, a diastatic enzyme inlet, and a fermentation microorganism inlet; heating means for adjusting a temperature inside the reaction vessel; pH control means for controlling pH inside the reaction vessel; stirring means for stirring a liquid inside the reaction vessel; and distillation means for distilling the liquid inside the reaction vessel, the distillation means being directly connected to the reaction vessel.

Moreover, it is preferable for the ethanol producing apparatus according to the present invention to be arranged such that the distilling means at least includes a distillation column and an ethanol collecting tank, the distillation column being provided above the reaction vessel, and a recovery outlet provided on a top section of the column being connected to the ethanol collecting tank.

Further, it is preferable for the ethanol producing apparatus according to the present invention to be arranged such that the diastatic enzyme inlet is connected to a diastatic enzyme storage tank, and the fermentation microorganism inlet is connected to a fermentation microorganism storage tank.

Moreover, the present invention may be in the following mode. Namely, an ethanol producing process according to the present invention is a process including the steps of: performing enzymatic saccharification of pre-treated biomass raw material in a reaction system; performing ethanol fermentation of fermentable sugars obtained from the saccharified pre-treated biomass raw material in the same reaction zone as the enzymatic saccharification; and distilling ethanol directly off from a reaction treatment liquid in the reaction zone, so as to recover the ethanol. An ethanol producing apparatus according to the present invention includes: a reaction vessel having a biomass raw material inlet, a diastatic enzyme inlet connected to a diastatic enzyme storage tank, and a fermentation microorganism inlet connected to a fermentation microorganism storage tank, each of which are provided at an upper section of the reaction vessel; an ethanol distillation column, provided above the reaction vessel and connected directly to the reaction vessel; an ethanol collecting tank connected to a recovery outlet at a top section of the distillation column, the reaction vessel further including: heating means for adjusting a temperature inside the reaction vessel; pH control means for adjusting pH; and stirring means (also called “mixing means”).

For a fuller understanding of the nature and advantages of the invention, reference should be made to the ensuing detailed description taken in conjunction with the accompanying drawings.

### BRIEF DESCRIPTION OF DRAWINGS

FIG. 1

FIG. 1 is a cross sectional view schematically illustrating one example of an apparatus to be used in an ethanol producing process of the present invention.

FIG. 2

FIG. 2 is an explanatory view of steps in one example of a conventional ethanol producing process.

### REFERENCE SIGNS LIST

1 reaction vessel
2 distillation column
3 raw material storage tank
4 biomass raw material inlet
5 diastatic enzyme storage tank
6 diastatic enzyme inlet
7 fermentation microorganism storage tank
8 fermentation microorganism inlet
9 ethanol recovery outlet
10 cooling tube
11 ethanol collecting tank
12 propeller-type mixer
13 heating wire
14 pH-adjusting alkaline reagent tank
15 pH-adjusting acid reagent tank
16 pH chemical inlet
20 ethanol producing apparatus

### DESCRIPTION OF EMBODIMENTS

The present invention is described in detail with reference to the attached drawings. However, the present invention is not limited to this.

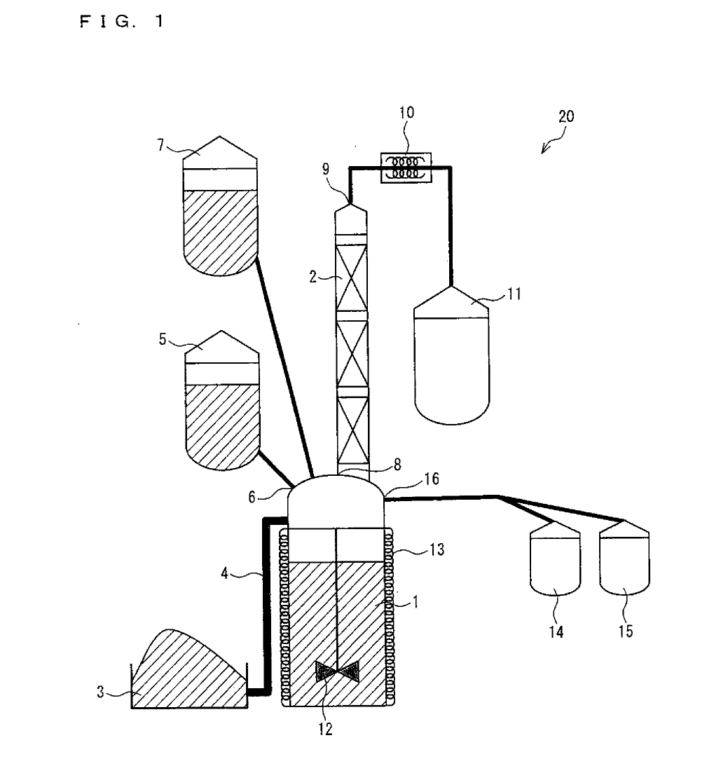

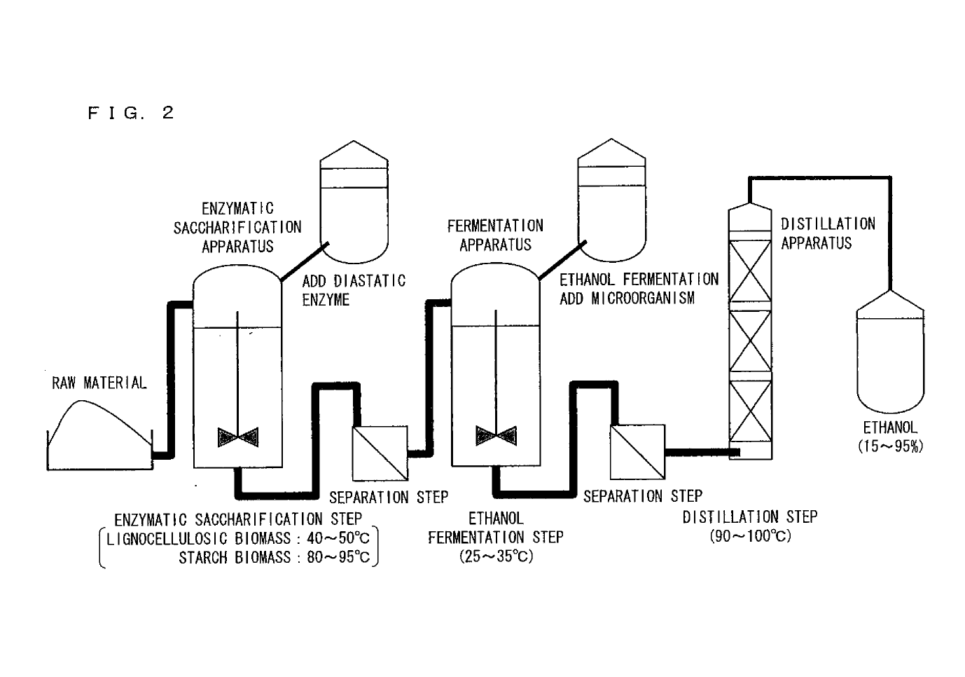

FIG. 1 is a cross sectional view schematically illustrating an arrangement of an ethanol producing apparatus 20 (hereinafter referred to as “apparatus 20”) that is suitable for conducting the ethanol producing process according to the present invention (hereinafter referred to as “process of the present invention”).

The apparatus 20 includes one reaction vessel 1 for carrying out saccharification and fermentation of biomass, and a distillation column 2 provided above and directly connected to a top section of the reaction vessel 1. An upper section of the reaction vessel 1 has a biomass raw material inlet 4 for feeding biomass raw material that is sent from a raw material storage tank 3, a diastatic enzyme inlet 6 for feeding diastatic enzyme from a diastatic enzyme storage tank 5, and a fermentation microorganism inlet 8 for feeding an ethanol-fermentable microorganism from a fermentation microorganism storage tank 7. An ethanol recovery outlet 9 provided on a top section of the distillation column 2 is connected to an ethanol collecting tank 11 via a cooling tube 10.

The distillation column 2 is means for carrying out rectification (fractional distillation), and is, for example, a fractionating column, a rectifying column, or the like. Providing the distillation column 2 to the apparatus 20 allows attainment of an effect such that vaporized ethanol and water are fractionally distilled inside the distillation column, which ethanol becomes concentrated as the ethanol moves towards an upper part of the tube, such that the ethanol is preferentially recovered.

In the apparatus 20, although the biomass raw material inlet 4, the diastatic enzyme inlet 6, and the fermentation microorganism inlet 8 are provided in the upper part of the reaction vessel 1, where these members are to be provided are not particularly limited. However, it is preferable for the biomass raw material inlet 4, the diastatic enzyme inlet 6, and the fermentation microorganism inlet 8 to be provided in the upper section of the reaction vessel 1. This is because the biomass raw material, the diastatic enzyme, and the fermentation microorganisms are fed into the reaction vessel 1 by gravity; thus, such an arrangement requires no specific feeding means such as a pump or the like to be provided to the apparatus 20. The “upper section” of the reaction vessel 1 denotes an upper half of the reaction vessel 1 in a state in which the reaction vessel 1 is placed by having a bottom surface of the reaction vessel 1 face downwards. With the apparatus 20, it is further preferable for the biomass raw material inlet 4, the diastatic enzyme inlet 6, and the fermentation microorganism inlet 8 to be provided at a position higher than a liquid surface of a liquid inside the reaction vessel 1. This is because such an arrangement does not require consideration of a case where the liquid flows out via the biomass raw material inlet 4, the diastatic enzyme inlet 6, and the fermentation microorganism inlet 8, and thus avoids the liquid in the reaction vessel 1 to mix with the biomass raw material, the diastatic enzyme, and the fermentation microorganism.

In addition, the reaction vessel 1 includes stirring means (also referred to as “mixing means”), for example a propeller-type mixer 12. The propeller-type mixer 12 stirs the biomass raw material and the diastatic enzyme evenly in the reaction vessel 1. This improves saccharification efficiency of the biomass raw material. Moreover, the propeller-type mixer 12 evenly stirs (i) saccharified solution generated in the enzymatic saccharification and (ii) the fermentation microorganism, and allows carrying out of aeration to the fermentation microorganism. This improves efficiency of the ethanol fermentation. The stirring means in the present invention is not limited to the propeller-type mixer, and other well known stirring means such as a magnetic stirrer or the like can be used as appropriate.

Heating means for adjusting an inside temperature is provided around the reaction vessel 1, for example a heating wire 13. The heating wire 13 is used as a heat source when carrying out (i) enzymatic saccharification to the biomass raw material, (ii) ethanol fermentation by use of the fermentation microorganism, and (iii) ethanol distillation. The heating means in the present invention is not limited to the heating wire, and well known heating means such as an immersion heater or the like may be used as appropriate.

Furthermore, in the reaction vessel 1, a pH chemical inlet 16 is provided for adjusting pH of the liquid inside the reaction vessel 1. The pH of the liquid inside the reaction vessel 1 is controlled to be in a desired range by adding a chemical into the reaction vessel 1 from a pH-adjusting alkaline reagent tank 14 and a pH-adjusting acid reagent tank 15, each of which are connected to the pH chemical inlet 16. More specifically, a computer receives data from pH measuring means (not illustrated) that is provided to the reaction vessel 1; this computer controls a feeding amount of the chemicals from the pH-adjusting alkaline reagent tank 14 and the pH-adjusting acid reagent tank 15, so as to control the pH of the liquid inside the reaction vessel 1 to the desired range. Note that, in the present invention, the pH control is not limited to the control by use of the computer as described above, and the amount of the chemicals fed from the pH-adjusting alkaline reagent tank 14 and the pH-adjusting acid reagent tank 15 may be controlled by an operator of the apparatus 20, so as to control the pH of the liquid inside the reaction vessel 1 to the desired range.

A process of the present invention includes the steps of: a first stage (enzymatic saccharification step) of storing, for 24 to 96 hours in a reaction vessel, (i) biomass raw material that is fed from the raw material storage tank 3 via a biomass raw material inlet 4 and (ii) diastatic enzyme that is fed from a diastatic enzyme storage tank 5 via a diastatic enzyme inlet 6, under a condition of a temperature in a range of 30° C. to 60° C. (preferably in a range of 40° C. to 55° C., most preferably in a range of 45° C. to 50° C.) and a pH in a range of 4 to 6 (preferably in a range of pH 4.5 to 5.0); a second stage (ethanol fermentation step) of storing, for 24 to 96 hours, a reaction product thus obtained and an ethanol-fermentable microorganism added together, under a condition of a temperature in a range of 20° C. to 40° C. (preferably in a range of 25° C. to 35° C., most preferably in a range of 28° C. to 30° C.) and a pH in a range of 4 to 7 (preferably in a range of pH 4.5 to 5.5, most preferably pH 5.0); and a third stage (distillation step) of distilling a reaction product thus obtained for 15 minutes to 12 hours under a condition of (i) a temperature in a range of 80° C. to 110° C. (preferably in a range of 90° C. to 105° C., most preferably in a range of 95° C. to 100° C.) under normal pressure, or (ii) a temperature in a range of 60° C. to 100° C. (preferably in a range of 80° C. to 95° C.) under reduced pressure (under pressure conditions lower than air pressure, preferably not higher than 800 hPa, more preferably not higher than 100 hPa), so as to distill and recover ethanol having a concentration in a range of 15% to 90% by volume. After carrying out these stages, raw material residue and microorganism residue remain in the reaction vessel as distillation waste and slurry. These raw material residue and microorganism residue can be discharged from a removal outlet (not illustrated), once all of the stages have terminated. The distillation is not limited to single distillation, and may be carried out as multistage distillation. This is because the multistage distillation is capable of acquiring ethanol of a higher purity. In a case where the distillation is carried out under the reduced pressure, that is, when vacuum distillation is to be carried out, a vacuum pump is provided to the apparatus 20.

On the other hand, raw ethanol that is recovered in the third stage (distillation step) is concentrated, if necessary, by further carrying out the distillation process or by use of various separation films, so as to obtain a concentrated ethanol having a concentration of 95% by volume or more.

It is preferable to carry out the pH adjustment in the first stage (enzymatic saccharification step) and the second stage (ethanol fermentation step) by adding the raw material, water, and the pH-adjusting agents as appropriate, so as to automatically control the pH to a range which is suitable for the reactions, in each of the steps.

It is preferable to automatically control in each of the stages, by use of a computer, (i) the reaction conditions such as a feeding amount and pH of the raw material, temperature, and mixing velocity, and (ii) concentration conditions of produced ethanol. However, the control of these various conditions may be manually carried out by an operator.

The biomass that is used as the raw material in the process of the present invention is lignocellulosic biomass. The “lignocellulosic biomass” encompass, for example: wood, wastepaper, rice straw, wheat straw, bagasse, and corn stover. These biomass are made of cellulose consisting of β1-4-linked glucose units, hemicellulose whose main component is xylose or mannose, and lignin. Hence, it is required for the biomass to be subjected to pre-treatment, so as to separate the lignin and the cellulose, and to crush the biomass.

The following for example may be carried out as the pre-treatment of the raw material: treatment by use of acid, alkaline, peroxide, or an organic solvent; coarse or fine crushing treatment by use of a cutter, a ball mill, or the like; crushing treatment by use of a press; blasting treatment; vapor or pressurized thermal water treatment; and supercritical water treatment.

The acid to be used for the acid treatment encompass, for example: sulfuric acid, hydrochloric acid, acetic acid, formic acid, phosphoric acid, oxalic acid, sulfur dioxide, and chlorine.

The alkaline to be used for the alkaline treatment encompass, for example: sodium hydroxide, calcium hydroxide, or ammonium. The peroxide to be used for the peroxide treatment encompass, for example: hydrogen peroxide, ozone, or perchloric acid. The organic solvent to be used for the treatment by use of the organic solvent encompass, for example: ethanol, ether, acetone, or dimethylformamide. A method for separating the lignin and the other components in the biomass are all well known, and the process of the present invention can select and use one of these well known methods as desired.

As the pre-treatment of the raw material, for example, Patent Literatures 5 to 7 disclose methods which recover a monosaccharide from biomass by use of, in high concentration, strong acids such as sulfuric acid and hydrochloric acid. Patent Literatures 8 to 10 disclose methods for producing a monosaccharide by utilizing hydrothermal reaction. Patent Literature 11 discloses a method for producing a sugar composition from biomass, which includes a step of treating biomass by use of more than two types of acid treatment liquids that have different acid concentration. Patent Literature 12 discloses a method for saccharifying biomass cellulose, which includes enzymatic treatment, hydrothermal treatment by use of peroxide (may contain aluminum phosphate in some cases), and ozone treatment. Moreover, Patent Literature 13 includes a description concerning monosaccharification by high-pressure cooking of bagasse.

Patent Literature 5

Japanese Patent Application Publication, Tokuhyohei, No. 11-506934 A (Publication Date: Jun. 22, 1999)

Patent Literature 6

Japanese Patent Application Publication, Tokukai, No. 2000-50900 A (Publication Date: Feb. 22, 2000)

Patent Literature 7

Japanese Patent Application Publication, Tokukai, No. 2006-101829 A (Publication Date: Apr. 20, 2006)

Patent Literature 8

Japanese Patent Application Publication, Tokukai, No. 2005-168335 A (Publication Date: Jun. 30, 2005)

Patent Literature 9

Japanese Patent Application Publication, Tokukai, No. 2006-136263 A (Publication Date: Jun. 1, 2006)

Patent Literature 10

Japanese Patent Application Publication, Tokukai, No. 2001-262162 A (Publication Date: Sep. 26, 2001)

Patent Literature 11

Japanese Patent Application Publication, Tokukai, No. 2007-89573 A (Publication Date: Apr. 12, 2007)

Patent Literature 12

Japanese Patent Application Publication, Tokukai, No. 2007-74992 A (Publication Date: Mar. 29, 2007)

Patent Literature 13

Japanese Patent Application Publication, Tokukai, No. 2000-50840 A (Publication Date: Feb. 22, 2000)

It is preferable for the lignocellulosic biomass that has been subjected to the crushing treatment or the like to have a particle diameter of not more than 2 mm (further preferably not more than 1 mm, most preferably not more than 0.2 mm). Whether or not the lignocellulose biomass is smaller than the preferable particle diameter can be determined by having the lignocellulose biomass pass through a mesh with openings of 2.0 mm (not more than 1.0 mm, or not more than 0.2 mm).

The process of the present invention may use starch biomass instead of the lignocellulosic biomass, as the raw material. The starch biomass encompass, for example: cereals such as rice, root vegetable, corn, and wheat; and commercial food waste which contain these components. This biomass is made of amylose consisting of α1-4-linked glucose units and amylopectin consisting of α1-6-linked amylase short chains. Thus, the biomass require the pre-treatment such as crushing and thermal treatment.

The enzyme to be used for saccharifying the pre-treated lignocellulose biomass in the first stage (enzymatic saccharification step) of the process of the present invention encompass, for example: cellulase, hemicellulase, pectinase, and a combination thereof. The enzyme to be used for saccharifying the starch biomass in the first stage encompass, for example: α-amylase, β-amylase, glucoamylase, hemicellulase, and a combination thereof. The saccharification in the first stage (enzymatic saccharification step) generates, from cellulose and hemicellulose, saccharides such as glucose, mannose, xylose, galactose, and arabinose. Lignin, on the other hand, substantially does not dissolve in water and is therefore not saccharified; thus, the lignin remains as saccharification residue. Note that, as the foregoing enzymes, commercialized enzyme reagents can be used as appropriate.

Next, as the ethanol-fermentable microorganism to be used in the second step (ethanol fermentation step) in the process of the present invention, a fermentation microorganism that is usually used in ethanol fermentation is to be used, such as: yeast such as Saccharomyces cerevisiae; amylo germs such as Mucor rouxii and Rhizopus delemar; and bacteria such as Zymomonas mobilis. It is also possible to use amylo germs which have both a saccharification effect and a fermentation effect, or a combination of such amylo germs and yeast. Microorganisms that are distributed from a microorganism depositary institution are usable as the ethanol-fermentable microorganism.

Other microorganisms may also be used in which (i) a fermentation ability has been newly added, (ii) a new type of saccharides have been newly added as a substrate, or (iii) effects have been reinforced, each of which are attained by carrying out crossing treatment, mutation treatment, or genetic recombination to a microorganism, such as ethanol-fermentable recombinant Escherichia coli or xylose-fermentable recombinant yeast.

An optimum condition in accordance with the ethanol-fermentable microorganism to be used is adopted as the condition of the ethanol fermentation in the second stage (ethanol fermentation step) of the process of the present invention, as appropriate.

The following Examples explain a best mode for carrying out the present invention, however the present invention is not limited to these Examples.

### EXAMPLES

### Example 1

### (1) Preparation of Raw Material

Cypress wood chips which are one type of the lignocellulosic biomass were subjected to fine crushing treatment by use of a ball mill, so as to obtain fine powder having an average particle diameter of 20 μm to 50 μm.

### (2) Pre-Treatment and Enzymatic Saccharification Step

400 g of the foregoing cypress wood fine powder (moisture content of 7%) was put into an ethanol producing apparatus illustrated in FIG. 1, and 2 liters of deionized water was added to this cypress wood fine powder. A heater was used so as to keep this mixture at a temperature of 45° C., and pH was adjusted to 5.0 by use of 6N sodium hydroxide and 6N hydrochloric acid. This mixture was mixed at a velocity of 250 rpm, which prepared a solvent. These temperature, pH and mixing velocity were controlled by an automated controlling apparatus.

Next, 10.4 g of cellulase (“Acremonium Cellulase” produced by Meiji Seika Kaisha, Ltd.) and 2 g of hemicellulase (“Y-2NC” produced by Yakult Pharmaceutical Industry Co., Ltd.) were added to the solvent, and this mixture was reacted for 72 hours while the above condition was maintained. This obtained an enzymatic saccharification liquid containing 143 g of glucose and 32 g of mannose.

### (3) Ethanol Fermentation Step

400 ml of yeast culturing liquid, which was obtained by aerobically culturing a commercial baker's yeast at 30° C. in a YPD liquid culture (containing 2% glucose, 2% polypeptone, and 1% yeast extract, and having a pH of 5.0), was added to the enzymatic saccharification liquid thus prepared in (2), in a reaction vessel. This mixture was ethanol fermented for 48 hours while stirred at 150 rpm, under a condition of a temperature at 30° C. and a pH of 5.0. This obtained an ethanol fermentation liquid having an ethanol concentration of 4.61% (v/v). This concentration is equivalent to 110.6 ml of pure ethanol.

### (4) Ethanol Distillation Step

A temperature inside the reaction vessel was risen to 95° C. while stirring the liquid at 250 rpm. Once this temperature was reached, the inside of the reaction vessel was kept at this temperature for 1 hour, so as to distill ethanol via a distillation tube provided above a top section of the reaction vessel and connected to a cooling tube thus connected to the distillation tube. This recovered an ethanol concentrated liquid of 420 ml.

An ethanol concentration of the ethanol concentrated liquid thus obtained was 24.3% (v/v). This concentration is equivalent to 102.1 ml of pure ethanol.

### (5) Recovery of Residue and Residual Liquid

Residue that remained in the reaction container after the distillation and recovery of the ethanol contained, as its main component, dead yeast, denatured enzyme protein, and lignin derived from the cypress fine powder. This residue had a property of aggregating upon application of heat. Therefore, once the mixing was stopped, the residue easily separated into solid and liquid. Such separated residual liquid contained, as its main component, water-soluble lignin, organic acids, culture medium component, yeast extract component, and residual ethanol [approximately 0.47% (v/v)]. These solid residue and residual liquid were separately taken out and recovered from different removal outlets. The residual liquid can be used for methane fermentation for example, and the solid residue, after washing with water and then drying, may be used as fuel, for example.

A yield of the ethanol from the raw material fine powder (moisture content of 7%) in this Example was 274 ml per 1 kg. A yield of ethanol from the saccharification fermentation liquid was 90.4%.

### Example 2

### (1) Preparation of Raw Material

High-quality wastepaper (a mixture of printing paper, publishing paper, copying paper and the like), which is one type of the lignocellulosic biomass, was made into paper chips having an average size of 5 mm×3 cm, by use of a shredder.

### (2) Pre-Treatment and Enzymatic Saccharification Step

2 kg of the foregoing paper chips were put into an ethanol producing apparatus illustrated in FIG. 1, and 19 liters of deionized water were added to the paper chips. A heater was used so as to keep this mixture at a temperature of 45° C., and pH was adjusted to 5.0 by use of 6N hydrochloric acid. This mixture was mixed at a velocity of 250 rpm, which prepared a solvent. These temperature, pH and mixing velocity were controlled by an automated controlling apparatus.

Next, 78 g of cellulase (“Acremonium Cellulase” produced by Meiji Seika Kaisha, Ltd.) was added to the solvent, and this mixture was reacted for 72 hours while the above condition was maintained. This obtained an enzymatic saccharification liquid containing 588 g of glucose and 153 g of xylose.

### (3) Ethanol Fermentation Step

Next, 30 g of commercial dried baker's yeast was added to the enzymatic saccharification liquid thus prepared in (2), inside the reaction vessel. This mixture was ethanol fermented for 48 hours while stirred at 150 rpm, under a condition of a temperature at 30° C. and a pH of 5.0. This obtained an ethanol fermentation liquid having an ethanol concentration of 2.0% (v/v). This concentration is equivalent to 380 ml of pure ethanol.

### (4) Ethanol Distillation Step

A temperature inside the reaction vessel was risen to 95° C. while stirring the liquid at 250 rpm. Once this temperature was reached, the inside of the reaction vessel was kept at this temperature for 5 hours, so as to distill ethanol via a distillation tube provided above a top section of the reaction vessel and connected to a cooling tube thus connected to the distillation tube. This recovered an ethanol concentrated liquid of 530 ml.

The ethanol concentration of the ethanol concentrated liquid thus obtained was 53.8% (v/v). This concentration is equivalent to 285 ml of pure ethanol.

### (5) Recovery of Residue and Residual Liquid

Residue that remained in the reaction container after the distillation and recovery of the ethanol contained, as its main component, dead yeast, denatured enzyme protein, water-resistant film used in processing of paper, and lignin-containing clay substance. Residual liquid that was separated from the residue contained, as its main component, soluble lignin, organic acids, culture medium component, yeast extract component, and residual ethanol [approximately 1.2% (v/v)]. These solid residue and residual liquid were separately taken out and recovered from different removal outlets. The residual liquid can be used for methane fermentation for example, and the solid residue, after washing with water and then drying, may be used as fuel, for example.

The yield of ethanol from the high-quality wastepaper raw material in this Example was 190 ml per 1 kg. The yield of ethanol from the fermentation liquid was 75%.

According to the present invention, the processes of the three stages are carried out in the same reaction vessel. Thus, it is possible to simplify and tightly arrange the apparatus and the producing steps. This is advantageous in that thermal energy is reduced.

Moreover, the residue that remains after termination of the process is aggregated due to application of heat. This allows easy separation of waste fluid and the residue, which also allows omission of the separation operations carried out in each step. Thus, no intermediate products are lost, and the yield of ethanol from the raw material is remarkably increased. Moreover, an effect is attained such that, an accident caused by clogging of a connection pipe, which occurs in a case where separate apparatuses are used, does not occur.

Further, production efficiency improves since highly-concentrated material can be used. As a result, this allows downsizing of the apparatus, and reduces costs of the product.

The embodiments and concrete examples of implementation discussed in the foregoing detailed explanation serve solely to illustrate the technical details of the present invention, which should not be narrowly interpreted within the limits of such embodiments and concrete examples, but rather may be applied in many variations within the spirit of the present invention, provided such variations do not exceed the scope of the patent claims set forth below.

### INDUSTRIAL APPLICABILITY

The invention is suitably used for producing industrial ethanol and fuel ethanol, from various types of biomass raw material.

1. An ethanol producing process comprising the steps of:
performing enzymatic saccharification of pre-treated lignocellulosic biomass in a reaction system;
performing ethanol fermentation of fermentable sugars obtained from the saccharified pre-treated lignocellulosic biomass in the same reaction zone as the enzymatic saccharification; and
distilling ethanol directly off from a reaction treatment liquid in the reaction zone, so as to recover the ethanol.

2. The ethanol producing process as set forth in claim 1, wherein:
the enzymatic saccharification of the pre-treated lignocellulosic biomass is carried out in the presence of a diastatic enzyme at a temperature in a range of 30° C. to 60° C.;
the ethanol fermentation of fermentable sugars obtained from the saccharified pre-treated lignocellulosic biomass is carried out in the presence of an ethanol-fermentable microorganism at a temperature in a range of 20° C. to 40° C. in the same reaction zone as the enzymatic saccharification; and
the ethanol distillation is carried out under normal pressure or reduced pressure at a temperature in a range of 80° C. to 110° C.

3. An ethanol producing apparatus comprising:
one reaction vessel having a biomass raw material inlet, a diastatic enzyme inlet, and a fermentation microorganism inlet;
heating means for adjusting a temperature inside the reaction vessel;
pH control means for controlling pH inside the reaction vessel;
stirring means for stirring a liquid inside the reaction vessel; and
distillation means for distilling the liquid inside the reaction vessel,
the distillation means being directly connected to the reaction vessel.

4. The apparatus as set forth in claim 3, wherein:
the distillation means at least includes a distillation column and an ethanol collecting tank,
the distillation column being provided above the reaction vessel, and a recovery outlet provided on a top section of the column being connected to the ethanol collecting tank.

5. The ethanol producing apparatus as set forth in claim 3, wherein:
the diastatic enzyme inlet is connected to a diastatic enzyme storage tank; and
the fermentation microorganism inlet is connected to a fermentation microorganism storage tank.

6. The ethanol producing apparatus as set forth in claim 4, wherein:
the diastatic enzyme inlet is connected to a diastatic enzyme storage tank; and
the fermentation microorganism inlet is connected to a fermentation microorganism storage tank.
